(19) United States
(12) Patent Application Publication
(10) Pub. No.: US 2008/0195024 A1
(43) Pub. Date: Aug. 14, 2008

(54) Treating Disorders Associated with Inflammation

(76) Inventors: Geert W. Schmid-Schonbein, Del Mar, CA (US); Daniel Fainman, San Diego, CA (US); Stephen F. Flaim, San Diego, CA (US); Wyming Lee Pang, Shoreline, WA (US); Alexander H. Penn, San Diego, CA (US)

Correspondence Address: FISH & RICHARDSON, PC, P.O. BOX 1022, MINNEAPOLIS, MN, 55440-1022, US

(21) Appl. No.: 12/018,026

(22) Filed: Jan. 22, 2008

Related U.S. Application Data
(63) Continuation-in-part of application No. PCT/US2006/028107, filed on Jul. 20, 2006.
(60) Provisional application No. 60/701,366, filed on Jul. 20, 2005.
(60) Provisional application No. 60/701,375, filed on Jul. 20, 2005.

(51) Int. Cl. A61M 1/34

(52) U.S. Cl. 604/6.09; 604/540

(57) ABSTRACT

Methods and apparatuses for treating or preventing disorders related to inflammation are provided. In the methods and apparatuses, a glass fiber filter or other filter is used to remove mediators of inflammation from a bodily fluid.

### CROSS-REFERENCE TO RELATED APPLICATIONS

This application is a continuation-in-part and claims benefit under 35 U.S.C. § 120 of International Application No. PCT/US2006/028107, having an International Filing Date of Jul. 20, 2006, which claims the benefit of priority from U.S. Provisional Patent Application Ser. Nos. 60/701,366, and 60/701,375, both filed Jul. 20, 2005. These applications are incorporated herein by reference in their entirety.

### TECHNICAL FIELD

This application relates to treatment of inflammation-mediated disorders.

### BACKGROUND

Inflammation plays a critical role in many diseases, illnesses, and disorders, such as asthma, arthritis, cancer, and heart disease. Mediators of inflammation are central to many processes, including pain, fever, and infection, and at least some of these mediators are involved in circulatory collapse and other signs heralding physiologic shock. In addition to inflammatory mediators such as cytokines and chemokines, products of enzymatic degradation of autologous tissue (e.g., lipid fragments and protein fragments) also can serve as inflammatory mediators. For example, lipids or lipid fragments and proteins or protein fragments are capable of launching or sustaining an inflammatory response or cascade, either directly or indirectly. See, e.g., Waldo et al. (2003) Shock 20:138-143. Such inflammatory fragments can mediate circulatory collapse and to increase cell death, among other effects.

### SUMMARY

Blocking formation of, removing, or otherwise controlling mediators of inflammation may be beneficial to subjects experiencing the ill effects of an inflammatory cascade. For example, controlling inflammatory mediators can be a useful method to control or cure shock or other inflammation-based disorders.

Thus, in one aspect this document provides a method for treating disorders related to inflammation. The method can include identifying a subject as having or being at risk for an inflammatory disease that is amenable to extracorporeal filtration of a bodily fluid; inserting a catheter (e.g., a venous catheter) into a selected body part of the subject; withdrawing the bodily fluid through the catheter; filtering the bodily fluid to remove one or more mediators of inflammation; and returning the filtered bodily fluid to the subject. The bodily fluid can be blood, lymph, cerebrospinal fluid, or peritoneal fluid. The bodily fluid can be circulated through and filtered in an extracorporeal filtration device. The filtering step can include passing the bodily fluid through one or more glass fiber filters (e.g., four or more glass fiber filters, or six or more glass fiber filters). The filtered bodily fluid can be returned to the subject through the catheter inserted into the selected body part. The method can further include a step for separating the blood into cellular and non-cellular components. The non-cellular component can be filtered and the cellular component can remain not filtered. The method can further include recombining the filtered non-cellular component with the unfiltered cellular component prior to returning the blood to the subject. The inflammatory mediators can be selected from the group consisting of polypeptides, polypeptide fragments, lipids, and lipid fragments. The subject can be diagnosed as being in shock, or can be diagnosed as having hypertension, diabetes, retinopathy, or Alzheimer's disease.

In another aspect, this document provides an apparatus for treating disorders related to inflammation. The apparatus can include a unit for removing a bodily fluid (e.g., blood, intraperitoneal fluid, cerebrospinal fluid, or lymph) from a subject, a unit for extracorporeal filtration of the bodily fluid, wherein the extracorporeal filtration unit is operable to remove mediators of inflammation from the bodily fluid, and wherein the mediators of inflammation comprise polypeptides, polypeptide fragments, lipids, or lipid fragments, and a unit for returning the filtered fluid to the subject. The filtration unit can include one or more glass fiber filters. The apparatus can further include a unit for separation of blood into cellular and non-cellular components.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows four representative examples of neutrophils after exposure to buffer (top row) or intestinal homogenate digested with chymotrypsin for 10 minutes (2nd row), 20 minutes (3rd row), or 30 minutes (bottom row). Arrows indicate locations of blebs. Scale at upper left=10 microns.

FIG. 2 is a graph plotting a time course of neutrophil cell death after exposure to protease digests of intestinal homogenates. Time zero represents the initial time of exposure to neutrophils. Intestinal homogenates were digested with trypsin, chymotrypsin, or elastase, as indicated. PBS was used as control. *: P<0.05 vs. control. #: P<0.05 vs. trypsin. Bars represent mean±SE; N=6 for each group.

FIG. 3 is a series of graphs plotting cell death (left column) and forward scatter (right column) of neutrophils incubated with protease digested intestinal wall homogenates (top row: chymotrypsin, middle row: elastase, bottom row: trypsin), versus reaction time with neutrophils. Graphs in the left column show the same data as in FIG. 2, but the data are broken down to show results for individual homogenates.

FIG. 4 is a pair of graphs plotting the time course of forward light scattering of neutrophils after exposure to individual intestinal wall homogenates diluted 1:1 (v/v) with PBS (N=6) (top), and selected cell activators (fMLF and PMA) or pancreatic proteases alone (N=4 for PBS, N=3 for the others; bottom). Bars represent mean±SD. *P<0.02 vs. PBS.

FIG. 5 is a series of micrographs of five neutrophils fixed after 20, 30, 60, or 90 minutes of incubation with intestinal wall controls (intestinal wall homogenates mixed with an equal volume of PBS). Neutrophils were stained with crystal violet and viewed at 1000× with light microscopy. Scale at upper left=10 microns.

FIG. 6 is a graph plotting protease activities measured in Relative Fluorescent Units (RFU) of pancreatic proteases mixed with equal volumes of buffer or intestinal wall after digestion. The protease activity of each group was significantly different from all other groups (P<0.02), except for the pairing marked not significant (NS). Bars represent mean±SD; N=6 for all groups.

FIG. 7 is a pair of graphs plotting cell death measured by PI labeling of neutrophils (top) and protease activity (bottom) of intestinal homogenates digested with chymotrypsin with addition of protease inhibitor (PMSF) or solvent at the beginning or end of the digestion period. Bars represent mean±SD; N=6. *: P<0.005, **: P<10⁻⁵.

FIG. 8 is a pair of graphs showing the effect of filtration on the cytotoxic activity of chymotrypsin digested intestinal wall homogenates. Top: cell death of neutrophils after a 30 minute exposure to filtered or unfiltered intestinal wall digested with chymotrypsin for 6 hours at 37° C. Bottom: protease activity of filtered and unfiltered digested homogenates. Bars represent mean±SD; N=6. *: P<0.003.

FIG. 9 is a graph plotting cell death after 20, 30, or 40 minutes of exposure of neutrophils to homogenates of rat small intestinal wall (Wall), luminal contents (Lumen), luminal contents filtered with glass fiber pre-filters (Filtered Lumen), intestinal wall digested with luminal contents (Wall+Lumen), and intestinal wall digested with filtered luminal contents (Wall+Filtered Lumen). Bars represent mean±SD; N=5. *: P<0.04 vs. Wall. #: P<0.05 vs. Lumen. ‡: P<0.03 vs. Filtered Lumen.

FIG. 10 is a graph plotting protease activities of homogenates of small intestine (Wall), luminal contents of small intestine (Lumen), luminal contents filtered with glass fiber pre-filters (Filtered Lumen), intestinal wall digested with luminal contents (Wall+Lumen), and intestinal wall digested with filtered luminal contents (Wall+Filtered Lumen). Bars represent mean±SD; N=3.

FIG. 11 is a graph plotting the effect of PMSF and ANGD pretreatment on protease activity in intestinal wall homogenates digested by lumen homogenates. The fraction of protease activity remaining is defined as the protease activity of the mixed homogenates plus inhibitor divided by the protease activity of the mixed homogenates plus buffer. Bars represent mean±SD: N=4. All pairs were significantly different (P<0.01).

FIG. 12 is a pair of graphs plotting neutrophil cell death (top) and forward light scatter (bottom) after exposure for 20, 30, or 40 minutes to intestinal wall homogenates digested with filtered luminal fluid. Bars represent mean±SD; N=4. Top: *: P<0.05 vs. controls. #: P<0.05 vs. ANGD. ‡: P<0.05 vs. PMSF. Bottom: *: P<0.01 vs. controls. (At 40 minutes, only one sample in the control and PMSF groups had enough cells living to measure forward scatter, thus no standard deviations or significant differences for those groups were computed.)

FIG. 13 is a graph plotting neutrophil cell death (top) and forward light scatter (bottom) after exposure to homogenates of rat food pellet mixed with equal volumes of PBS or digested with chymotrypsin or luminal fluid, as indicated. Luminal fluids at the same dilution and incubation times were used for another control. Bars represent mean±SD; N=1, 2, 4, and 4 from left to right.

FIG. 14 is a graph plotting neutrophil activation by PBS (PRE), rat pancreatic homogenate (RPH), or shocked plasma that was filtered for 2, 4, or 8 minutes, as indicated.

FIGS. 15A-15D are a series of graphs plotting neutrophil activation by PBS, (PRE), RPH, or shocked plasma that was filtered through 3, 4, 6, or 7 glass fiber filters (GFF) in series, as indicated.

FIG. 16 is a graph plotting mean arterial blood pressure in rats transfused with 5 (n=1), 7 (n=3), 8 (n=1), or 9 (n=1) ml normal blood.

FIG. 17 is a graph plotting mean arterial blood pressure in rats transfused with 7 ml SAO shock blood.

### DETAILED DESCRIPTION

Shock and other disorders are associated with a rise in levels of inflammatory mediators found in blood and other bodily fluids. These inflammatory mediators trigger a cascade of inflammation that can cause, for example, the hypotension and multi-organ failure that are hallmarks of shock. Inflammatory mediators also appear to play a role in certain infectious diseases and chronic illnesses.

Although the source and triggers of these mediators have eluded investigators, evidence suggests that peritoneal fluid can act as a pool of inflammatory mediators, which can be delivered into neighboring tissues and into the central lymphatic and blood circulation. The mediators can enter the peritoneal fluid through the intestinal wall, perhaps under the influence of pancreatic proteases. See, e.g., Penn and Schmid-Schoenbein, “Evidence for the Creation of Cytotoxic Factors by Proteases in the Intestinal Lumen: A Possible Damage Mechanism in Shock,” presented at the Shock Society meeting, June 2003; and Ishimaru et al. (2004) Shock 22:467-471.

Compounds that block the production or effects of these and other mediators of inflammation have not proven effective in controlling shock or other pathological effects of these mediators. As provided herein, however, mediators or their triggers (e.g., lipids, lipid fragments, proteins, and protein fragments) may be filtered or subjected to other methods of removal from body tissues and fluids such as blood, lymph, cerebrospinal fluid, or peritoneal fluid. The removal of inflammatory mediators or their triggers may prevent, reduce, or arrest activation of the inflammatory cascade and prevent or treat the consequences of inflammation and underlying disorders.

This document provides methods and devices for removing or reducing the quantity of inflammatory mediators contained in bodily fluids and/or tissues. For example, methods can include identification of a subject having or at risk for an inflammatory disease or condition that is amenable to extracorporeal bodily fluid filtration. Such diseases and conditions can include acute disorders such as physiologic shock, or chronic diseases such as hypertension, diabetes, retinopathy, or Alzheimer's disease. Identification of subjects suffering from or at risk for one or more of these ailments can proceed according to customary diagnostic processes, including the use of clinical signs and symptoms and laboratory tests.

Once identified, the subject can be prepared for extracorporeal bodily fluid filtration by establishing suitable access through a catheter. After insertion of an indwelling catheter, for example, blood (e.g., femoral, brachial, or venous blood) or another bodily fluid can be withdrawn from the subject and circulated through an extracorporeal filtering device (e.g., a dialysis or apheresis device).

After the blood or other bodily fluid has been withdrawn into the extracorporeal filtering device, it can be filtered to remove selected substances that are involved in the inflammatory process or cascade (i.e., inflammatory mediators or triggers), such as lipids, lipid fragments, proteins, and protein fragments. Removing the selected substances can reduce the effects of the inflammatory process by attenuating or halting cell activation and the inflammatory cascade, thus attenuating or halting the adverse effects associated with the inflammatory cascade.

In some embodiments, an extracorporeal filtering device can be an apheresis system. Apheresis systems for removing from the blood molecules such as low-density lipoprotein (LDL) can safely and effectively lower the level of LDL cholesterol in humans, and have been applied to the treatment of certain forms of hypercholesterolemia.

In some embodiments, an extracorporeal filtering device can be similar to a peritoneal dialysis system, in which a solution is run through a tube into a subject's peritoneal cavity and then drained by gravity. The drained fluid then can be filtered and returned to the peritoneal cavity.

In some embodiments, cerebrospinal fluid can be passed through and filtered in an extracorporeal filtering device. Inflammation and mediators of inflammation are important components of several acute and chronic central nervous system (CNS) disorders. Preventing, reducing, or blocking the inflammatory cascade in peripheral blood might be of less value in the treatment of such disorders, as the blood-brain barrier prevents the free flow of components from peripheral blood into the CNS. Thus, direct filtration of an affected subject's cerebrospinal fluid might be the most effective way to eliminate mediators of inflammation and to attenuate their effects.

Any suitable filter or filtration system can be used to remove inflammatory mediators and their triggers (e.g., lipids, lipid fragments, proteins, and protein fragments). Glass fiber filters may be particularly useful. For example, a glass fiber syringe pre-filter manufactured by Pall Gelman (East Hills, N.Y.) can be used. Such filters can absorb cytotoxic factors in digested organ homogenates, as described herein. Even glass fiber filters with relatively open pore structures (e.g., 1-40 micron pore size) can be effective in removing cytotoxic factors. Filters containing any other suitable material, including hydrophobic polymers (e.g., nitrocellulose) or other hydrophobic surfaces, also can be useful. A bodily fluid can be filtered through one filter, or through more than one filter (e.g., 2, 3, 4, 5, 6, 7, 8, 9, 10, or more than 10 filters). In some embodiments, for example, a bodily fluid (e.g., whole blood, plasma, cerebrospinal fluid, intraperitoneal fluid, or lymph) can be passed through a series of two or more filters prior to being returned to the subject from which it was removed.

Commercially available and known filtration systems also can be useful. For example, an LDL-apheresis system such as a dextran-sulfate cellulose (DSC) system, a heparin-induced LDL precipitation (HELP) system, an immunoadsorption system, or a direct adsorption of lipoproteins hemoperfusion (DALI) system can be used.

The methods provided herein can include filtration of whole blood or, optionally, a separation procedure can be included. For example, red blood cells cannot pass through glass fiber filters. Thus, whole blood can be separated into cellular and non-cellular (e.g., plasma) components prior to filtration. In some embodiments, non-cellular components can be filtered to remove inflammatory mediators, and then can be recombined with cellular components. In some embodiments, cellular components can be filtered and then recombined with unfiltered non-cellular components, or both cellular and non-cellular components can be filtered separately and then recombined.

Other procedures also can be performed when carrying out the methods described herein. For example, in some embodiments an extracorporeal filtration process may include addition of fluid or other materials (e.g., albumin) to filtered fluid before it is returned to the body. In some embodiments, the complement cascade can be suppressed to avoid unwanted activation of the complement system. For example, a serine protease inhibitor can be added to the filtrate during filtration to suppress activation of the complement system. In some embodiments, an extracorporeal filtration process can include one or more steps to inhibit or reduce protease activity that may be activated within the bodily fluid before or during the filtration process. For example, a solution of one or more (e.g., 1, 2, 3, 4, 5, 6, 7, 8, 9, 10, or more than 10) protease inhibitors, such as L-(tosylamido-2-phenyl)ethyl chloromethyl ketone (TPCK), 1-chloro-3-tosylamido-7-amino-2-heptanone (TLCK), pepstatin, leupeptin, chymostatin, antipain, aprotinin, phenylmethylsulfonyl fluoride (PMSF), and (4-amidino-phenyl)-methane-sulfonyl fluoride (APMSF) and 6-amidino-2-naphtyl p-guanidinobenzoate dimethanesulfate (ANGD, nafamostat mesilate) can be added to a bodily fluid prior to or during filtration (e.g., after removal from the body, or concomitant with filtration. Alternatively or in addition, a filter can be coated with or otherwise contacted by one or more protease inhibitors prior to the filtration step of a method provided herein.

Filtration can occur for any suitable length of time, and can be applied to any suitable volume of blood or other bodily fluid. The removed and filtered fluid is returned to the subject, and the removal and filtration process can begin again immediately or at any suitable point in the future. Thus, the filtration process can be applied to fluid from the same subject repeatedly over the course of days, weeks, or years.

This document also provides devices for implementing the methods described herein. An apparatus can include a fluid (e.g., blood or intraperitoneal fluid) removal unit, an extracorporeal filtration unit, and a filtered fluid return unit. The removal unit can be operable to remove blood or any other appropriate bodily fluid from a subject through a suitable device, such as an indwelling catheter. The filtration unit can be operable to filter out inflammatory mediators and/or their triggering agents, and can include any suitable filter, as described herein. In some embodiments, an apparatus for filtering blood can be coupled to or can include an optional separator unit for separating blood into its cellular and non-cellular components, so that the filtration can be applied to one or more separated blood components. A filtered fluid return unit can allow a subject's filtered bodily fluid to be returned to the body.

The invention will be further described in the following examples, which do not limit the scope of the invention described in the claims.

### EXAMPLES

### Example 1

### Materials and Methods for Examples 2-6

Materials: TPCK-treated trypsin, TLCK-treated chymotrypsin, and pancreatic elastase were obtained from Worthington Biochemicals (Lakewood, N.J.), and formyl-methionine-leucine-phenylalanine tripeptides (fMLF), phorbol myristate acetate (PMA), PMSF, dimethyl sulfoxide, ethanol, Dextran 229, Histopaque-1077, Percoll, and propidium iodide (PI) were obtained from Sigma Chemical Corp. (St. Louis, Mo.). 6-amidino-2-naphthyl p-guanidinobenzoate dimethanesulfonate (ANGD, Nafamostat Mesilate) was from Torii Pharmaceutical Co. (Tokyo, Japan), and hydrophobic borosilicate glass fiber filters (Pall Gelman; East Hills, N.Y.) were from Fisher Scientific (Pittsburg, Pa.).

In vitro studies of intestinal wall homogenate digested with proteases—surgical procedure and organ collection: Male Wistar rats were given general anesthesia (sodium pentobarbital, 50 mg/kg, i.m.) and cannulated via the left femoral vein. The animals were euthanized (120 mg/kg sodium pentobarbital, i.v.), and the small intestine was harvested, cut into 3 to 4 sections, and rinsed in cold phosphate buffered saline (PBS). The luminal contents (solid and semi-liquid content of the small intestine containing partially digested food and digestive enzymes, etc.) were removed by manual peristaltic compression. Intestinal sections were slit open longitudinally, placed in sealed centrifuge tubes with 40 ml of cold PBS, and agitated to remove residual luminal contents and/or digestive enzymes from the mucosal surface. Samples were then transferred to another tube with 40 ml of cold PBS and agitated again. The rinsed intestinal sections were placed into tubes, weighed, and frozen (−80° C.) until homogenization. For selected experiments the luminal contents were saved, weighed, and frozen for later homogenization.

In vitro studies of intestinal wall homogenate digested with proteases—tissue homogenization: Six milliliters of cold PBS per gram of tissue were added to frozen intestine or luminal content. The intestines and luminal contents were then homogenized over ice with a tissue homogenizer (Kinematica Polytron PT 1200C, Brinkmann, Westbury, N.Y.) for at least 5 minutes following disintegration of large tissue chunks. Samples were spun at 4000 g for 4 minutes at room temperature. Supernatants were collected and further centrifuged (16,000 g for 30 minutes, 4° C.), and passed through a single hydrophobic borosilicate glass fiber syringe filter to reduce tissue debris and remove colloidal materials. Filtered supernatants of intestinal walls (referred to herein as wall homogenate) and supernatants of luminal contents (luminal fluid) were divided into aliquots and stored (−80° C.) for later experiments.

In vitro studies of intestinal wall homogenate digested with proteases—enzymatic digestion of wall homogenates: Wall homogenates were rapidly thawed at 37° C. and mixed with equal volumes of PBS, TPCK-treated trypsin, TLCK-treated chymotrypsin, or elastase. Controls of the proteases alone also were created by mixing them with equal volumes of PBS. Since lower grades of trypsin are often contaminated with the activity of chymotrypsin and vice versa, trypsin pretreated with TPCK (a specific chymotrypsin inhibitor) and chymotrypsin pretreated with TLCK (a trypsin inhibitor) were used to reduce any effects of cross contamination. Before mixing with wall homogenate, the enzymes were reconstituted in PBS at the following concentrations: 1 mg/ml for TPCK-treated trypsin, 1 mg/ml for TLCK-treated chymotrypsin, and 0.5 mg/ml for elastase.

The mixtures were incubated at 37° C. for 3 or 6 hours depending on the experiment and again frozen (−80° C.) for later testing on neutrophils and for protease activity measurements. Digested wall homogenates (wall homogenates mixed with a protease) were more translucent than undigested wall homogenates (wall homogenates mixed with PBS) and upon settling have less sediment. In some samples, 10 μl of the general serine protease inhibitor PMSF was added to 400 μl of digested wall homogenate prior to or immediately following digestion (1 mM PMSF final). PMSF permanently inhibits serine enzymes, including trypsin, chymotrypsin, and elastase, but has a short half-life in aqueous solutions. PMSF reacts with water to become a volatile gas and evaporates after about an hour, thus making the solutions safe to mix with cells and, in principle, minimizing any direct effect PMSF may have on the cells. For selected studies, digested wall homogenates after incubation were passed through a series of four hydrophobic borosilicate glass fiber syringe filters.

In vitro studies of cell cytotoxicity mediated by luminal content—cytotoxicity of luminal fluid and digests of wall homogenate by luminal fluid: Three sample groups were formed to examine the ability of the luminal content in the intestine to create cytotoxic mediators:

luminal fluid alone (Lumen group),

wall homogenate alone (Wall group), and

wall homogenate digested by luminal fluid (Wall+Lumen group).

To investigate the effect of pre-filtering luminal fluid, two additional groups were formed:

filtered luminal fluid alone (Filtered Lumen group) and

wall homogenate digested with filtered luminal fluid (Wall+Filtered Lumen group).

The groups were treated as follows: the luminal fluids from 5 animals were incubated separately (37° C., 3 hours) and aliquots of each sample were collected. One aliquot was filtered through 4 glass fiber pre-filters in series. The unfiltered and filtered aliquots were mixed with equal volumes of PBS (to generate the Lumen and Filtered Lumen groups, respectively) or with wall homogenate from the same animal (to generate the Wall+Lumen and Wall+Filtered Lumen groups, respectively). The Wall group was prepared by mixing wall homogenates with equal volumes of PBS. All samples were incubated at 37° C. for 3 additional hours, aliquotted, and frozen (−70° C.) for later evaluation of neutrophil cytotoxicity and protease activity.

In vitro studies of cell cytotoxicity mediated by luminal content—PMSF and ANGD: Luminal fluid contains a mixture of digestive enzymes and cytotoxic mediators generated by these enzymes. To determine if enzyme inhibition could prevent the generation of cytotoxic mediators from luminal contents, luminal fluids were pre-filtered through three glass fiber filters in series, a procedure that reduces cytotoxic activity, allowing the experiments to be focused solely on new cytotoxic mediator formation. Following filtration, PMSF, ANGD, a combination of PMSF and ANGD, or buffer was added at room temperature. The luminal solutions with and without protease inhibitors were mixed with an equal volume of wall homogenate (final concentrations: 1 mM for PMSF and 1.25 mg/ml, i.e., 2.31 mM, for ANGD) and incubated at 37° C. for 3 hours. ANGD was not completely soluble in the luminal fluid (with or without glucose), and a white precipitate formed upon addition of the solubilized ANGD. The precipitate was separated following the digestion period and the supernatant collected. All samples were then aliquotted and frozen for later measurements of protease activity and neutrophil cytotoxicity.

In vitro studies of cell cytotoxicity mediated by luminal content—food pellet homogenate digested by chymotrypsin or enzymes in luminal fluid: Standard rat diet pellets (Harlan Teklad Rodent Diet W-8604) were homogenized and centrifuged using the same protocol as used for the intestinal wall (without filtration at the end). The supernatant of rat diet homogenate was mixed with equal volumes of PBS buffer, 1 mg/ml chymotrypsin, or luminal fluid from 4 different rats, and digested for 6 hours at 37° C. Samples were added to fresh un-stimulated neutrophils for 30 minutes and assayed for cytotoxicity.

Cytotoxicity measurement—neutrophil isolation: Fresh human neutrophils were isolated from heparinized whole blood with Percoll gradients and re-suspended in PBS at room temperature to a concentration of 2×10⁶ neutrophils per ml (Penn, “Digestive enzymes in the generation of cytotoxic mediators during shock.” Ph.D. Thesis, Department of Bioengineering, UCSD, La Jolla, Calif., 2005).

Cytotoxicity measurement—Neutrophil morphology: To determine cell morphology with light microscopy, 100 μl of isolated human neutrophils were mixed with 100 μl of PBS or 100 μl of wall homogenate digested with chymotrypsin for 6 hours at 37° C. After a 10-, 20-, or 30-minute incubation period, the cells were fixed by addition of 100 μl 3% glutaraldehyde (1% final) and then stained with crystal violet in 3% acetic acid.

Cytotoxicity measurement—flow cytometric analysis of neutrophil cytotoxicity: For measurements of cell death and forward scatter (a measure of cell “size”) by flow cytometry, 100 μl of sample were mixed with 100 μl of cells (10⁶ cells/ml final) and after selected periods of incubation, a life/death indicator (200 μl of 2 μM propidium iodide, PI) was added. Within seconds of PI addition, the sample was tested in a flow cytometer (Beckton-Dickson FACScan; Franklin Lakes N.J.).

Flow cytometric analysis of neutrophils was carried out by plotting forward scatter (FSC) on the ordinate and FL-2 (PI fluorescence) on the abscissa. The fluorescence of control cells in PBS buffer in FL-2 was the same as live cells without PI in the medium. Two regions on the scatter plot were gated. Region 1 (R1) was for live cells, and was constructed to monitor an increase in cell size that results from bleb formation and also to observe the small uptake of PI that occur may prior to total membrane failure. As cells died, their signal was simultaneously shifted upwards in FL-2 and downward in forward scatter, appearing as a second population in Region 2 (R2). Few cells fell into the transition region between the two populations. As cells became PI-positive, they moved from the first to the second population. Neutrophils in the process of dying after incubation with a digested wall homogenate also were observed, with dead cells in R2 and the remaining live cells at the far right in R1. Ten thousand cells were evaluated per sample. Cell death was reported as the percentage of total cells located in R2, i.e., Death=100%*(# in R2)/(# in R1+# in R2). It was notable that the cells that labeled PI-positive (i.e., “dead”), even in the absence of PI, retained their forward and side scatter position, forming a second population distinct from the PI-negative “live” cells. Tissue debris from the homogenate also was evident at the lower left and outside of the gates, and appeared even in the absence of cells.

Protease activity measurements: Proteolytic activity was determined using a serine protease activity kit (E6639 Enzcheck Protease Assay Kit, Molecular Probes). The substrate used for measuring protease activity was casein, derivatized with pH-insensitive fluorophores. Fluorescence was measured in triplicate using a spectrophotometer (Spectromax Gemini XS) with Softmax Pro software (Molecular Devices Corp., Sunnyvale, Calif.) and expressed as relative fluorescent units (RFUs). The fluorescence produced in a sample was related both to the number of sites in the casein molecule cleaved by the proteases in the sample and the turnover rate of the proteases. Proteases that act very rapidly, such as trypsin, chymotrypsin, and elastase, may approach a maximum within the timeframe of the assay indicating complete digestion of the substrate at the cleavage sites corresponding to that protease's specificity.

In each well, 16 μl of sample and 64 μl of digestion buffer were mixed with 80 μl of protease substrate solution at 37° C. In the study of digests of intestinal wall with pure proteases, fluorescence was measured after 90 minutes of digestion. In all other studies, measurements of fluorescence were made every minute for 60 minutes. Pilot studies showed that the fluorescence usually approached its maximum within that time. Therefore fluorescence values are reported at the 60-minute time point.

Statistics: Unless indicated otherwise, mean and standard deviations are shown. Differences between groups were calculated using the two-tailed paired student's T-test assuming unequal variances. P values are listed and p<0.05 was considered significant.

### Example 2

### Effects of Digested Wall Homogenates on Human Neutrophils

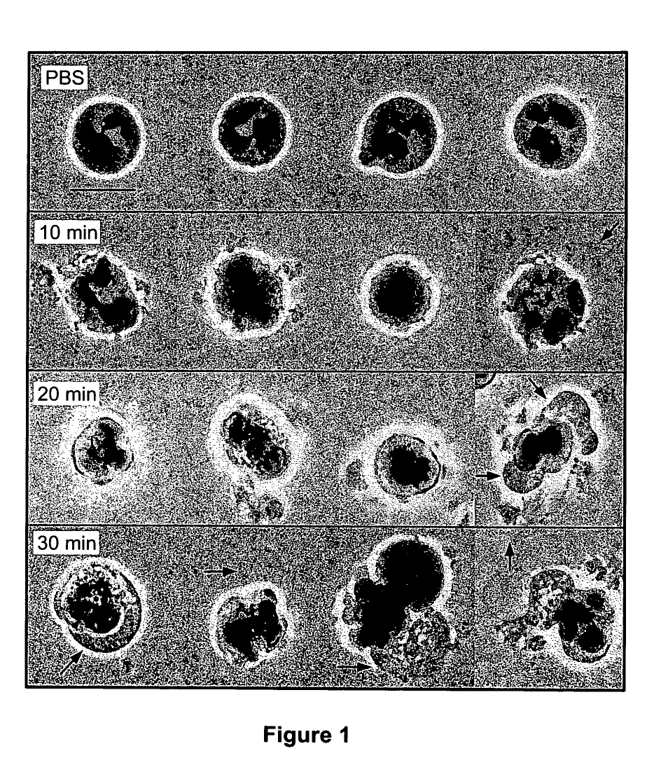

Morphology of neutrophils exposed to digests of intestinal wall: After addition of chymotrypsin-digested wall homogenate, individual neutrophils (FIG. 1) increased in cell size due to membrane bleb formation. In non-activated control neutrophils, crystal violet stained the nucleus blue while the cytoplasm was purple. After a 10 minute exposure to digested homogenate, irregular cytoplasmic extensions on the cell surface became visible as well as detectable blebbing. Blebs were observed forming on top of blebs, indicating that both mono- and bi-leaflet detachments may have been present (bottom-right cell, FIG. 1). Within a period of 30 minutes, the digested homogenate caused complete destruction of the internal cell structure.

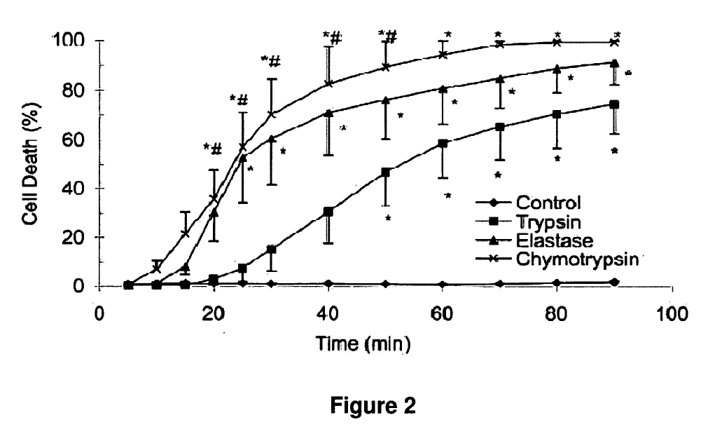

Cell death caused by digests of intestinal wall homogenates: Digests of wall homogenate with the pancreatic proteases trypsin, chymotrypsin, or elastase caused progressive neutrophil death over a period of 5 to 90 minutes of exposure. Cell death caused by chymotrypsin-digested wall homogenates was significantly enhanced compared to the effect of undigested wall homogenates after 20 minutes. Similarly, significant cell death occurred after 25 minutes for elastase digests and within 50 minutes for trypsin digests (FIG. 2). Though just as lethal in the long run, trypsin digested wall homogenate caused significantly (P<0.05) lower cell death than the chymotrypsin-digested wall homogenates at the early and middle times between 20 and 50 minutes. Death of cells in buffer, with or without proteases, or in wall homogenate alone remained below 2% at all times and within the range of experimental noise.

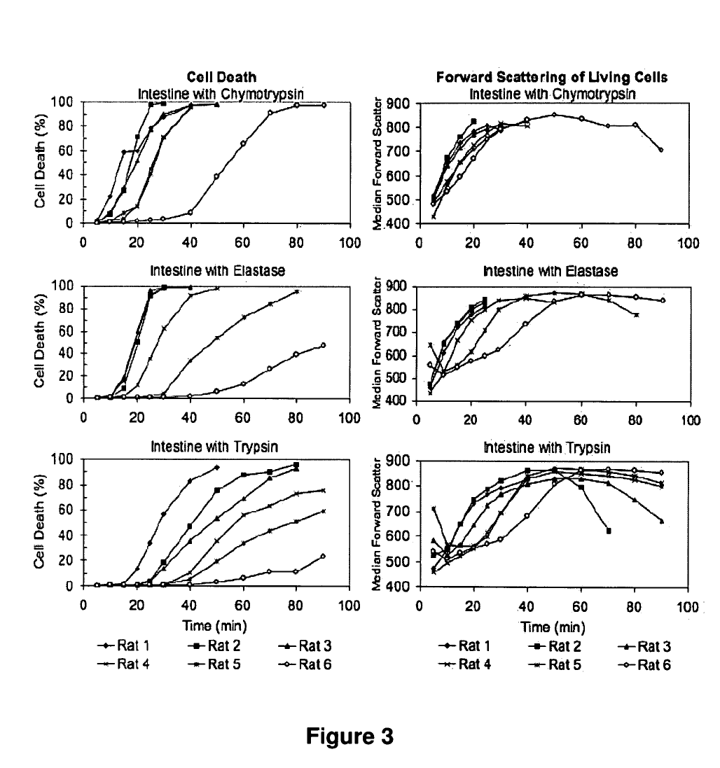

Although all digested wall homogenates caused cytotoxicity regardless of the choice of protease, differences were noted among rats based on rate of cell death (slope) and the time of onset (FIG. 3). For example, digested homogenate from rat #1 was more cytotoxic than digested homogenate from rat #6, irrespective of whether trypsin, chymotrypsin, or elastase was used. Also, for each protease, there was a time when digested homogenates from rats 1 or 2 gave over 95% cell death and homogenate from rat 6 gave less than 10%. Based on these time courses, digestion periods of 20, 30, and 40 minutes were used in later studies.

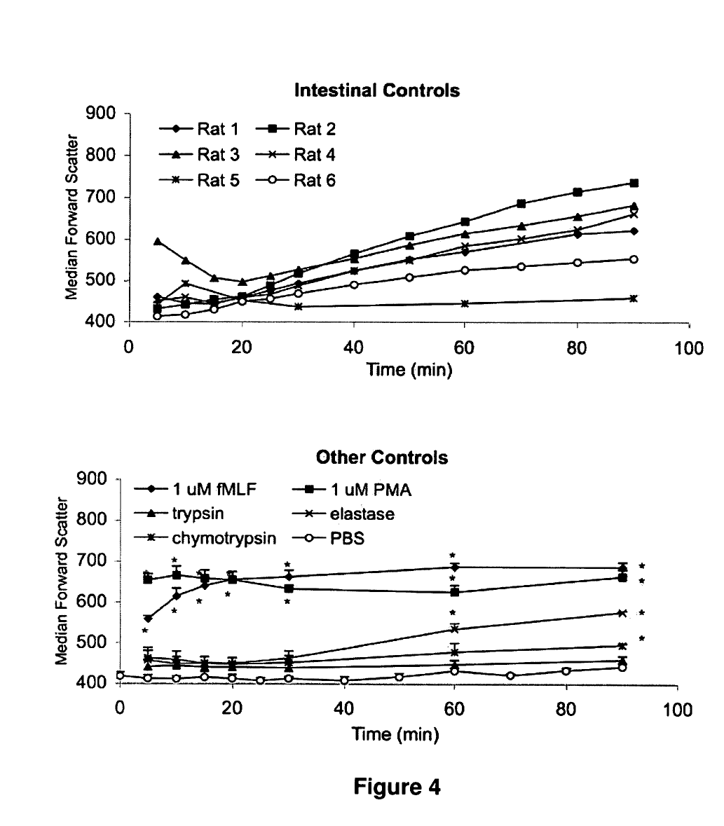

Forward scatter increases with digested wall homogenates: In all cases, an increase in neutrophil forward scatter was observed prior to cell death (FIG. 3, right side). This rise in forward scatter was significantly larger than that of cells activated by fMLF or PMA to produce pseudopods (FMLF and PMA results in FIG. 4, bottom; P<0.05 for fMLF vs. chymotrypsin digests by 20 minutes, and vs. trypsin and elastase digests at 60 minutes). When the neutrophils died, they exhibited decreased forward scatter and simultaneously increased in FL-2. These observations suggested that membrane blebs had formed, but had collapsed when membrane integrity failed and PI was allowed to enter the cell cytoplasm.

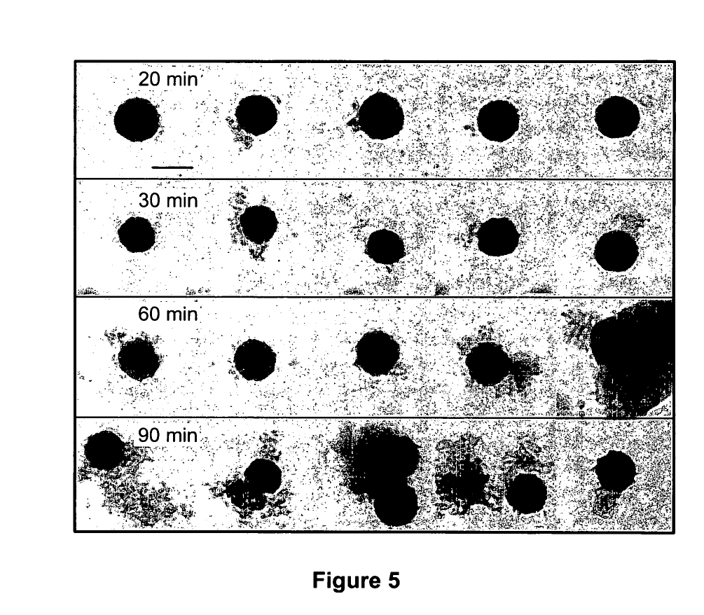

Forward scatter increase in controls: Though no cell death occurred with undigested wall homogenates, there was a gradual increase in forward scatter of neutrophils after exposure to these homogenates (FIG. 4, top). Light microscopy of cells exposed to undigested homogenates showed the cells remained round and of normal size, but as time progressed, many cells had debris from the homogenate attached to their membranes (FIG. 5). This may account for the cells' increase in forward scatter.

Pancreatic proteases alone caused relatively little increase in forward scatter (FIG. 5). Elastase caused the greatest increase (p<0.05 at 60 and 90 minutes). The chymotrypsin sample also became significantly elevated at 90 minutes. Trypsin had no significant effect on forward-scatter.

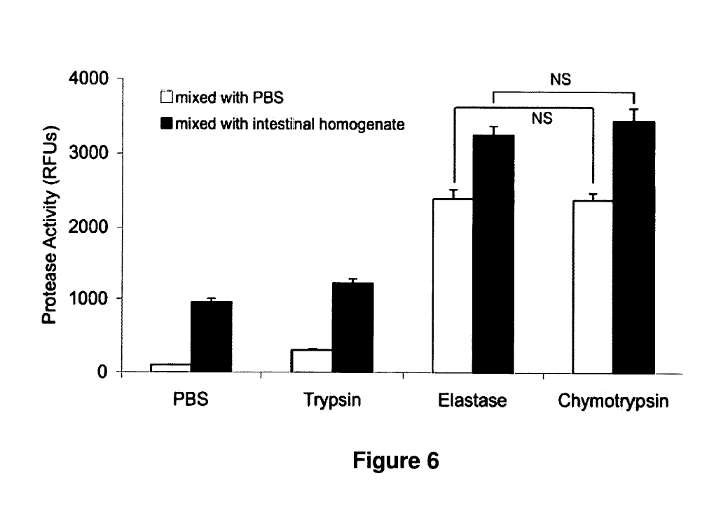

Proteolytic activity of digested intestinal wall and controls: Separate aliquots of digested and undigested wall homogenates and controls from the above cytotoxicity assay were tested for proteolytic activity (FIG. 6). The protease control values averaged 300±33 RFU for trypsin, 2371±230 RFU for chymotrypsin, and 2389±300 RFU for elastase. Even thoroughly rinsed, the wall homogenates from the rat retained high protease activity, averaging 953±136 RFU. For each protease, the combined protease activity of the wall homogenates and the exogenous protease was within one standard deviation of the protease activity for the corresponding digested wall homogenate. At the concentrations used, the individual pancreatic proteases rapidly reached their maximum fluorescence, i.e., further addition of the same protease did not increase the protease activity. Thus, if protease activity in the undigested homogenate was due to residual pancreatic trypsin, chymotrypsin, or elastase remaining after washing, one would have expected the protease activity in digested homogenate to be less than the combined activities of the undigested homogenate and the individual pancreatic protease due to the duplication of protease specificity in the latter two. The fact that the protease activities were directly additive suggests that the protease activity in the intestinal wall was not due to the presence of residual pancreatic proteases from the intestinal lumen, but rather came from proteases present in the intestinal wall tissue.

### Example 3

### Serine Protease Inhibition Prevents Cytotoxic Activity

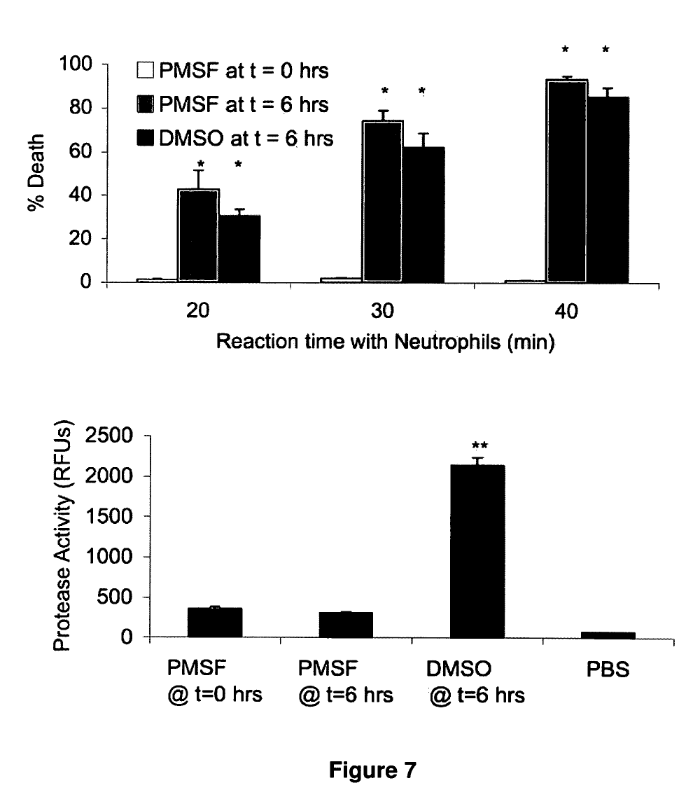

To determine whether cell death depended on protease action directly on the neutrophils, PMSF or buffer was added at the beginning or the end of the digestion period. PMSF inhibited the protease activity of the digested homogenate when added to wall homogenates before or after 6 hours digestion by chymotrypsin at 37° C. (FIG. 7, bottom). However, only the samples with PMSF added prior to digestion had no cell death (FIG. 7, top). The samples with PMSF added after digestion, if anything, slightly increased cytotoxicity compared to the samples without PMSF (not statistically significant).

### Example 4

### Glass Fiber Filtration Reduces Cytotoxic Activity

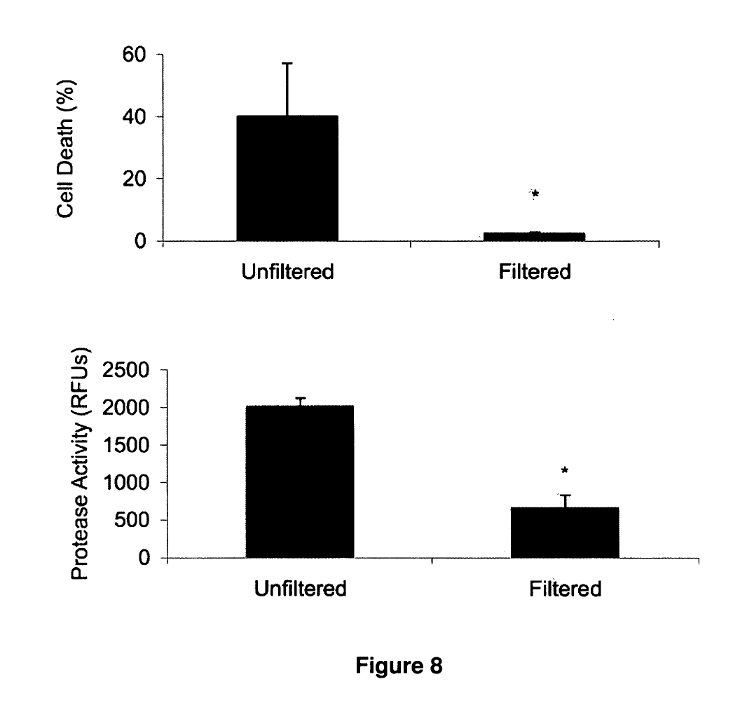

Passing digested wall homogenates through a hydrophobic glass fiber syringe pre-filter reduced cytotoxicity, an effect that was cumulative with repeated filtration. Filtering through four glass fiber syringe pre-filters in series eliminated cell death in neutrophils exposed for 30 minutes to wall homogenates that had been digested by chymotrypsin for 6 hours at 37° C. (top panel, FIG. 8). In pilot studies, filtering the supernatant of intestinal wall homogenate prior to digestion with protease did not noticeably affect the cytotoxicity after digestion, while filtration reduced the noise from homogenate debris in the flow cytometry. Thus, filtration was used as a final step in the preparation of wall homogenate.

While reduction in cell death was significantly reduced by filtration, protease activity was only partly eliminated (FIG. 8, bottom). Since filtration permitted partial retention of proteases, and may not remove other enzymes, cytotoxicity may be increased by further digestion when substrate remains in the wall homogenate.

### Example 5

### Luminal Content of the Intestine is a Source for Cytotoxic Factors

Luminal fluid contains cytotoxic factors and digestive enzymes: Unlike pure trypsin, chymotrypsin, and elastase, which at the tested concentrations were not cytotoxic to neutrophils, luminal fluid sometimes possessed cytotoxic activity. Thus experiments were carried out to distinguish cytotoxicity that was already present in luminal fluid from cytotoxicity caused by formation of new cytotoxic mediators after digestion of wall homogenate by luminal fluid.

Luminal fluid was incubated for 3 hours at 37° C. Aliquots were filtered with glass fiber (4 pre-filters in series). Incubation of luminal fluid, with or without filtering, prior to mixing and incubation with wall homogenate or PBS served two purposes. First, it increased the cytotoxic activity in the Lumen and Wall+Lumen groups by giving the enzymes in the lumen homogenate time to digest pre-cytotoxic substrate into cytotoxic factors. Secondly, by transforming pre-cytotoxic substrates into cytotoxic factors that were then susceptible to removal by filtration, it ensured that the final cytotoxicity in the Wall+Filtered Lumen group was due only to the action of the enzymes in the luminal fluid on the wall homogenate and not due to digestion of residual pre-cytotoxic substrate in the luminal fluid less susceptible to filtration.

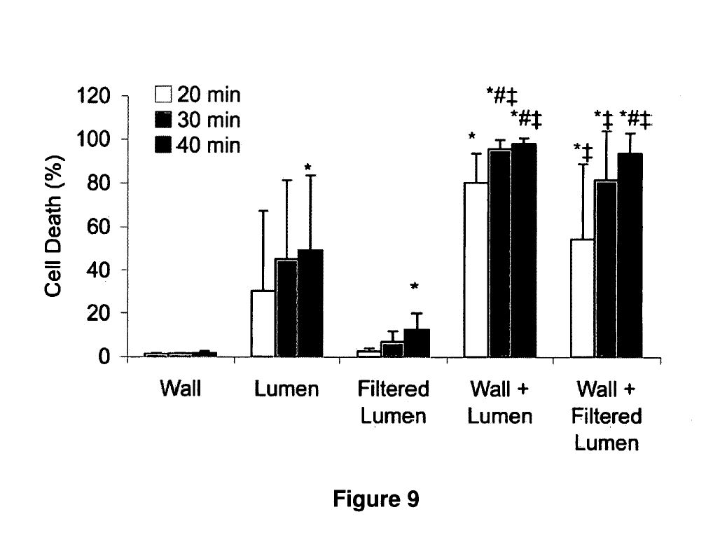

The Wall control group caused no cell death (FIG. 9). In contrast, the Lumen group caused cell death in 4 of 5 samples. Filtering luminal fluid (Filtered Lumen group) reduced cytotoxicity, but the results were not significant compared to the Lumen group (P<0.08 at 30 and 40 minutes). Digesting wall homogenate with unfiltered luminal fluid (Wall+Lumen group) resulted in significantly higher levels of cell death at all time points compared to Wall or Lumen groups alone. Cytotoxicity levels were greater than with wall homogenate digested by any one of the individual proteases (compared to results in FIG. 3; P<0.012 for Wall+Lumen vs. Wall+Chymotrypsin or Wall+Elastase at 20 minutes and P<0.004 vs. Wall+Trypsin at all three time points). Filtered luminal fluid also created strong cytotoxicity when incubated with wall homogenate (Wall+Filtered Lumen). Wall+Filtered Lumen was significantly more cytotoxic than Wall or Filtered Lumen groups alone, and was also significantly more cytotoxic than trypsin-digested intestinal wall homogenates at all time points (FIG. 2).

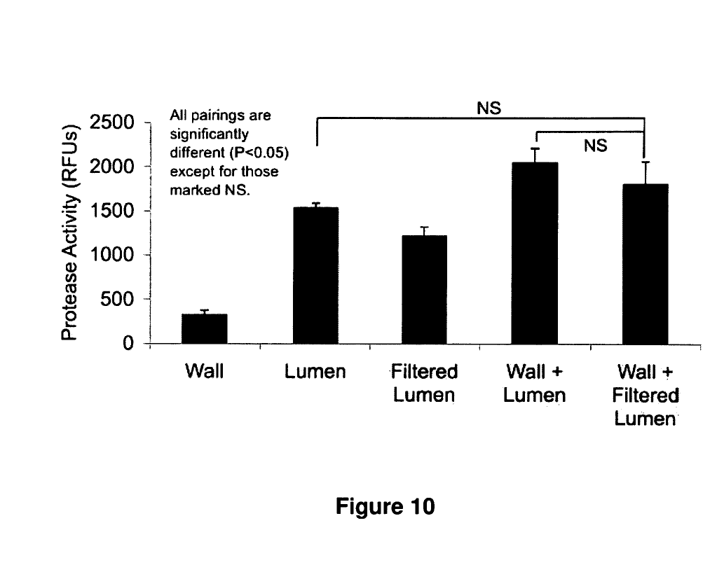

Proteolysis caused by luminal fluid: Protease activity of the Lumen group was greater than that of the Wall group (FIG. 10). Protease activity of the Wall+Lumen group was on average 10% higher than the sum of the protease activity of the two separate components, indicating activation of additional proteases or increased protease activity of already active proteases. Filtering luminal fluid resulted in only an average 20% drop in protease activity (Lumen vs. Filtered Lumen). There was a 16% enhancement in the protease activity of Wall+Filtered Lumen compared to the sum of the protease activities of Wall and Filtered Lumen.

Inhibition of digestive enzymes in luminal contents prevents cytotoxicity: Studies were conducted to determine whether addition of broad-spectrum digestive enzyme inhibitors to luminal fluid could prevent cytotoxicity in wall homogenates that were mixed with the luminal fluid. Combined with filtration, this might prevent most cytotoxicity.

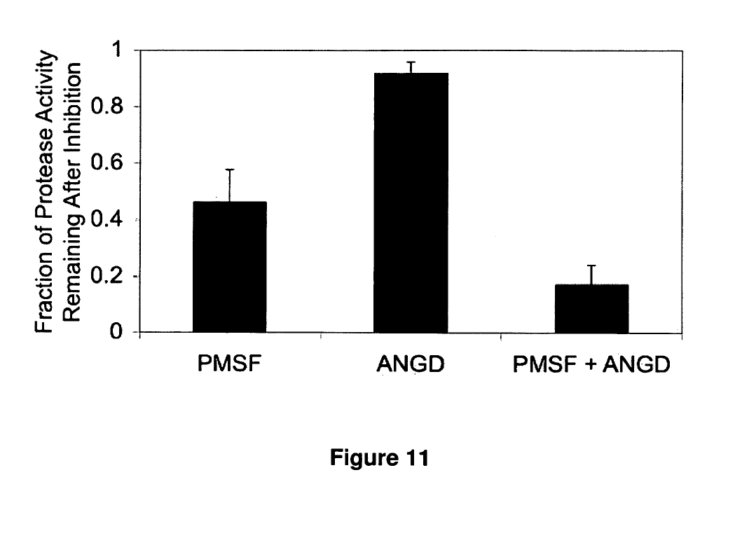

Luminal fluid was filtered, and PMSF, ANGD, PMSF+ANGD, or buffer was added before incubation with wall homogenates for 3 hours at 37° C. PMSF inhibited approximately half the protease activity in the digested homogenates, while ANGD was less effective. Combined, however, they inhibited the protease activity in the digested homogenates to less than 20% of controls with buffer alone (FIG. 11).

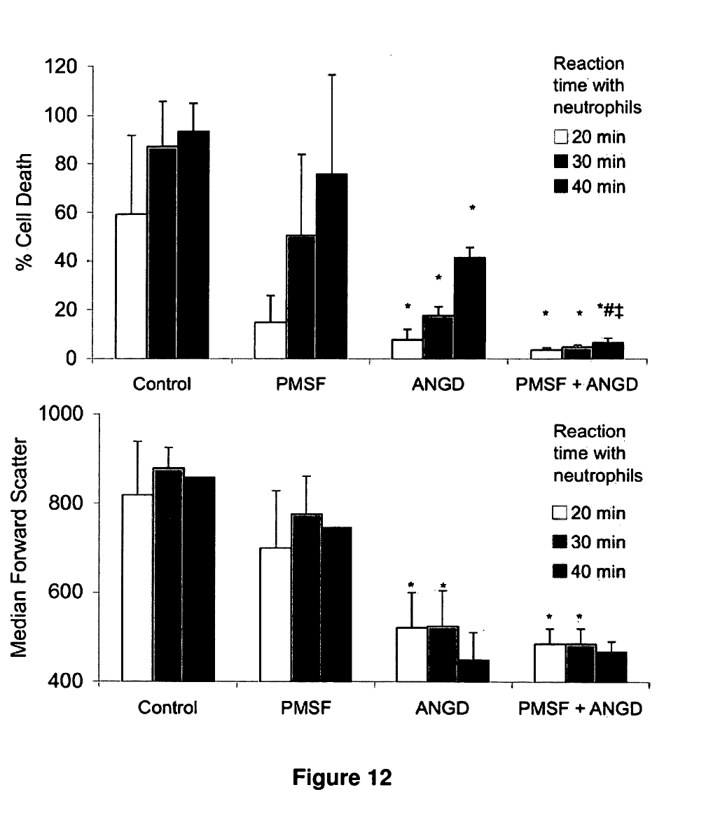

Neither PMSF nor ANGD alone entirely prevented cell death, although ANGD was able to significantly decrease the cell death compared to controls (FIG. 12, top). However, when ANGD was combined with PMSF, the two inhibitors completely prevented cell death. When forward scatters of the cells were examined (FIG. 12, bottom), it was noted that ANGD treated samples prevented the large increase in forward scatter that had been seen previously prior to cell death. The samples treated with both inhibitors had the same forward and side scatter values in flow cytometry as those of neutrophils in PBS, indicating that the neutrophils did not undergo a significant bleb or pseudopod formation.

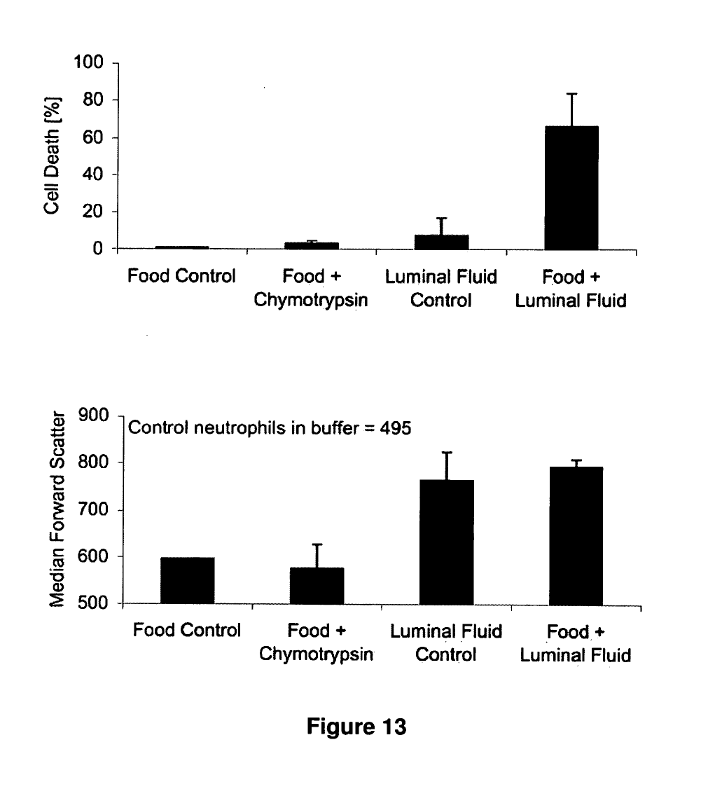

Digests of rat food by luminal fluid are cytotoxic: Digestion of rat food by luminal fluid but not by chymotrypsin alone or lumen homogenate control resulted in cytotoxic activity (FIG. 13; P<0.01). Chymotrypsin-digested food did not produce neutrophil cell death or activation greater than that caused by mixing with food alone. Forward scatter of cells exposed to lumen homogenate controls was significantly greater (P<0.05) than with food digested by chymotrypsin, suggesting that although cytotoxicity was present, a longer incubation time with neutrophils would be required to observe cell death. All groups showed increased forward scatter compared to un-reacted neutrophils.

Taken together, these studies indicated that there is a link between the permeability increase in the intestinal wall and the early stages of shock with formation of inflammatory and cytotoxic factors. These factors may either be present in the form of digested food in the intestinal lumen, or may be created by the action of digestive enzymes on interstitial structures after entering the intestinal wall, and may cause the intestinal necrosis observed in shock. These findings support the hypothesis that lavage of the content of the small intestine with broad-spectrum inhibitors may be protective in shock.

### Example 6

### In Vivo Studies

Experimental shock is induced in rats using one or more of several different methods. For example, hemorrhagic shock is induced by occluding the perimesenteric artery or by removal of blood from the femoral artery, whereas septic shock is induced by administration of endotoxin (typically at a dose of 3-5 mg/kg). Following shock induction, femoral blood is removed, filtered, and returned to the animals. Control animals either are not subjected to experimental shock or, if shock has been induced, are given blood that has been removed but not filtered. Animals are then monitored for effects of shock (e.g., death). In addition, blood samples are removed from control and experimental animals for measurement of inflammatory signals (e.g., levels of inflammatory mediators).

### Example 7

### Materials and Methods for Example 8 and 9

Human neutrophil isolation: To isolate human neutrophils for in-vitro evaluation, 30 mL of blood were collected in heparinized tubes and poured into 50 mL centrifuge tubes containing 3 mL of 6% dextran. The dextran solution was established by adding 6 g of Dextran 229 to 100 mL PBS. The contents of the 50 mL centrifuge tube were then mixed and the blood was allowed to sediment for 30-40 minutes. The plasma layer was then collected and gently placed upon at least 5 mL of Histopaque-1077 in a 50 mL centrifuge tube. This tube was centrifuged at 600 g for 20 minutes. All supernatant was removed, leaving a layer of granulocytes on a small layer of erythrocytes. This pellet was re-suspended in 5 mL PBS, and then gently transferred into a 15 mL centrifuge tube containing 3 mL of 55% percoll layered on top of 3 mL of 74% percoll. Maintaining the interface between the two percolls, the 5 mL of re-suspended pellet were gently placed on top of the 55% percoll layer. The 15 mL tube was centrifuged at 600 g for 15 minutes, using a slow deceleration rate. The layer of neutrophils was removed and transferred into a 15 mL centrifuge tube, and PBS was added. The 15 mL tube was centrifuged for 10 minutes at 100 g and the supernatant was removed. The clean neutrophils were re-suspended in PBS.

Rat pancreatic homogenate (RPH) preparation: Pancreases from healthy euthanized rats were harvested. Each organ was rinsed in cold 1×PBS for removal of blood and hair that may have accumulated during harvesting. Fat, mesentery, and intestinal connective tissue were trimmed off and the organ was rinsed again. The wet weight of the organ was established, and the pancreas was coarsely chopped using a razor blade and transferred to a 50 mL centrifuge tube. A volume of PBS 10 times the weight of the organ (in mL) was added to the centrifuge tube, and a homogenizer was applied at low speed for 10 minutes and then at high speed for 20 minutes. Ten μL of 1 mg/mL Trypsin was added to the homogenate, followed by incubation for 30-40 minutes at 37° C. To stop proteolytic reactions, the homogenate was placed on ice following incubation. Next, the homogenate was centrifuged for 5 minutes at 4000 g. The liquid fraction of the supernatant was removed and centrifuged for 30 minutes at 16,000 g at 4° C., and the pellet fraction was discarded. The liquid fraction of the supernatant was removed, stored on ice, and aliquoted as desired. This RPH served as a positive control for in-vitro experiments.

Optimization of filter flow rate: RPH, shocked plasma, or PBS were added to 5 mL syringes. Two glass fiber filters (GFF) in series were attached to the ends of the syringes. These syringes were then placed upon an automated syringe pump that was operated at 2 mL/min, 4 mL/min, or 8 mL/min. Filtrates were collected in 2.0 mL tubes and applied to human naïve neutrophils for evaluation by a neutrophil pseudopod projection assay.

Optimization of number of filters in series: RPH, shocked plasma, or PBS were added to 5 mL syringes. The ends of the syringes were coupled to three, four, six, or seven filters in series and placed on an automated syringe pump. The syringe pump was operated at 8 mL/min and the filtrates were collected in 2.0 mL tubes. The filtrates were applied to human naïve neutrophils that were analyzed for activation by pseudopod projection formation.

Neutrophil pseudopod projection assay: Sample or control volumes of 200 μL were placed into 2.0 mL tubes in triplicate. 200 μL of human naïve neutrophils were applied to each tube and the combination was incubated for 30 minutes at 37° C. 200 μL of 3% gluteraldehyde were then applied to each 2.0 mL tube. A preactivation control was established by placing 400 μL of human naïve neutrophils into 2.0 mL tubes in triplicate and then 200 μL of 3% gluteraldehyde were added to each tube. Seven μL of sample, control, or preactivation control were applied in triplicate to glass slides. Under light microscopy, these slides were analyzed for neutrophil activation. A cell count of at least 100 cells per sample was conducted and neutrophils were considered activated if a pseudopod projection beyond threshold was observed.

Animal studies: Male Wistar rats from Charles River Laboratories were utilized for animal studies. Animals were administered xylazine followed by nembutal for anesthesia purposes. Donor and acceptor animals were cannulated via femoral arteries and veins. In normal blood transfusion experiments (n=6), a central abdominal incision was performed to expose the abdominal aorta. At least 10 mL of blood were collected from the abdominal aorta into heparinized catheter connected syringes. Seven mL (an effective volume as determined by pilot studies) of donor blood were then transfused into an acceptor animal via femoral vein. An automated syringe pump insured a transfusion rate of 0.075 mL/min, an effective rate of transfusion as determined by pilot studies. This same protocol was followed for experiments where splanchnic arterial occlusion (SAO) shock blood was transfused into an acceptor. SAO shock was induced in male Wistar rats (n=9) by making a central abdominal incision and isolating the superior mesenteric and celiac arteries. Ischemia was achieved by clamping these arteries for 90-150 minutes. This was followed by a reperfusion period of 60-120 minutes initiated by removing the clamps from the arteries. This SAO shock blood was collected via abdominal aorta and transfused into an acceptor animal as described above. Mean arterial blood pressure was measured via pressure transducer connected through the femoral artery.

### Example 8

### Optimization of a Filtration Device for Removal of Inflammatory Mediators Released Upon Physiological Shock

Studies were conducted to optimize a filtration device utilized for removal of humoral inflammatory mediators released upon physiological shock, in an attempt to achieve maximal reduction/removal of cellular activating factors by GFF with minimal adverse effects on biological systems is the ultimate goal. Optimizing GFF-device performance included in vitro testing of filtration system parameters, including the number of GFFs in series and filtration flow-rate.

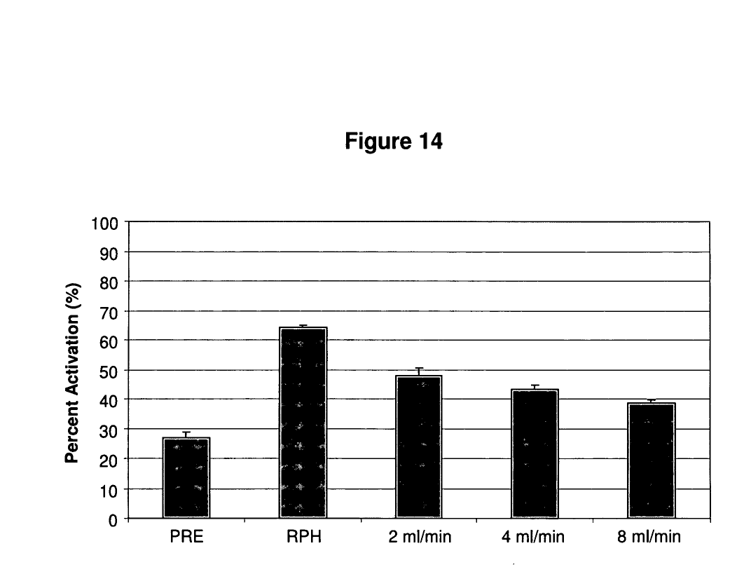

Through experimentation, filtration at higher filtration flow-rates results in better GFF-device performance. As illustrated in FIG. 14, filtration at 8 mL/min resulted in the greatest reduction of neutrophil activation. Overall, the percentage of neutophil activation was reduced to ˜39% with a filtration flow-rate of 8 mL/min compared to the percentage neutrophil activation observed for the positive control RPH, which was ˜64%. The percentage of neutrophil activation was 5% lower for this 8 mL/min flow-rate than for the 4 mL/min flow-rate, which had a neutrophil activation of 44%. When compared to the 2 mL/min flow-rate, which had a neutrophil activation of 48%, the 8 mL/min flow-rate was 9% lower. These experiments were conducted with two filters in series, which might account for the lack in dramatic activation reduction.

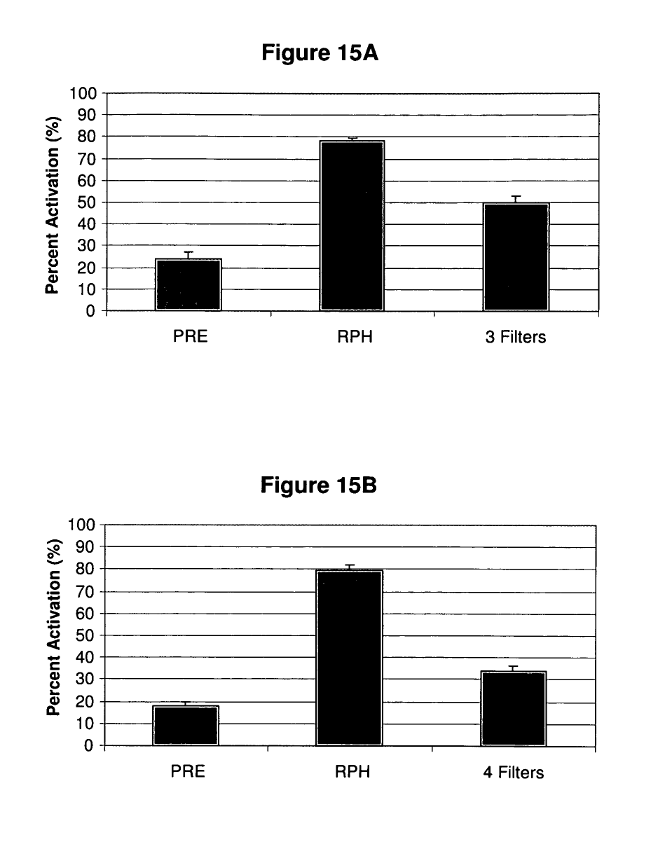

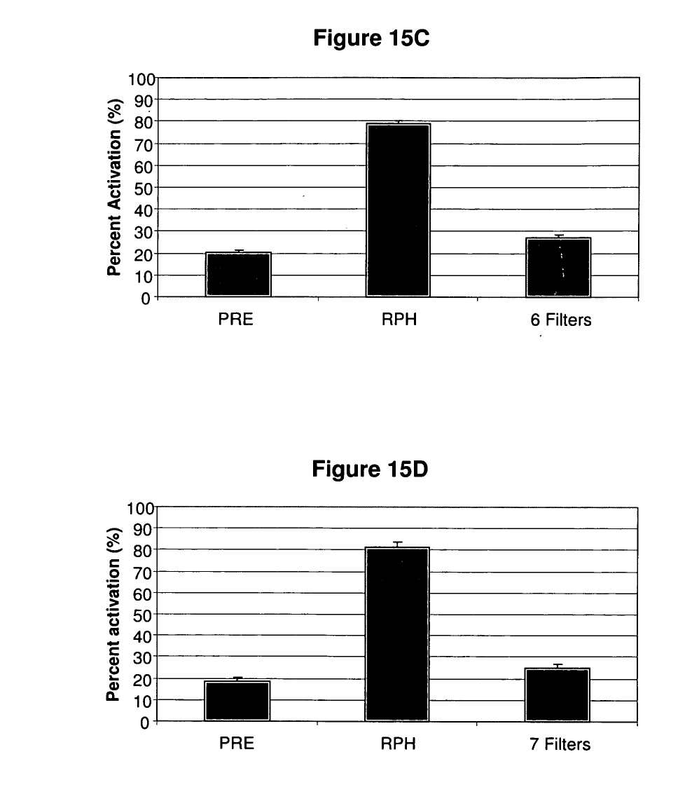

FIGS. 15A-15D illustrate the percentage of neutrophil activation when multiple filters were placed in series. Compared to the neutrophil activation percentage of the RPH positive control within each experiment, seven filters in series (FIG. 15D) demonstrated the greatest reduction in activation. Relative to each experiment's neutrophil activation percentage for positive control RPH, three filters in series (FIG. 15A) resulted in ˜29% reduction in activation, four filters in series (FIG. 15B) resulted in ˜48% reduction in activation, six filters in series (FIG. 15C) resulted in ˜52% reduction in activation, and seven filters in series (FIG. 15D) resulted in ˜56% reduction in activation. These filtrations were conducted at the 8 mL/min flow-rate, which the above-described experiment had shown to be optimal.

The optimal flow-rate experiments indicated that higher flow-rates make the GFF-device perform inflammatory mediator reduction/removal more efficiently. Although the results illustrated in FIG. 14 lack the dramatic reduction in neutrophil activation as observed in FIGS. 15A-15D, it is noted that the experiments in FIG. 14 were conducted with only two filters in series. Conducting the same experiment with more filters may result in greater reduction of neutrophil activation than that observed in FIG. 14. Although the GFF-device coupled to seven filters in series demonstrated the greatest reduction in percentage of activated neutrophils (FIG. 15D), the reduction was not significantly greater than that accomplished by six filters in series (FIG. 15C). Additionally, the retention of plasma volume with each added filter translates to less useable “clean” plasma collected after filtration by seven filters in series compared to six filters in series. The ability to retain more filtered plasma volume, combined with comparable filtration performance, indicates that six filters in series is a better design for the GFF-device than seven filters in series. Overall, these experiments demonstrated that design parameters for optimal GFF-device performance should include six filters in series and device operation at a filtration flow-rate of 8 mL/min.

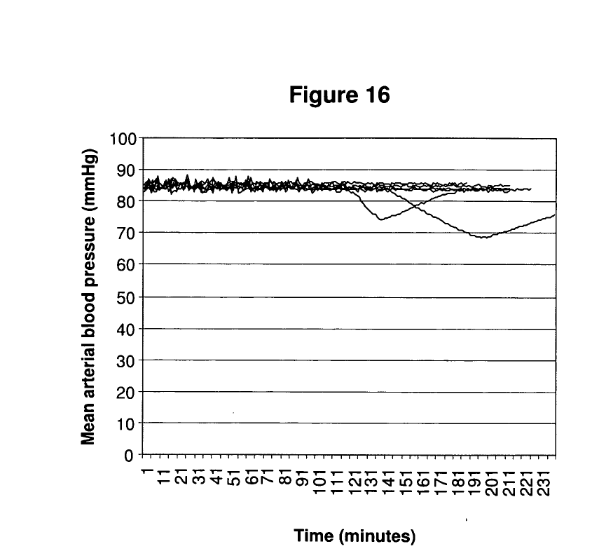

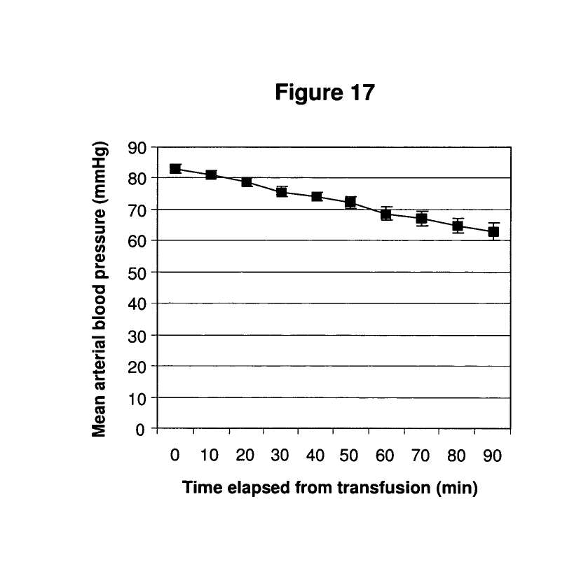

Comparing mean arterial blood pressure traces from acceptor animals transfused with normal blood (FIG. 16) with mean arterial blood pressure traces of acceptor animal transfused with SAO shock blood (FIG. 17), it became evident that SAO shock has a potentially lethal effect on biological systems. When comparing the normal blood transfusion animals that received seven mL of transfusion volume with the animals that underwent transfusion of equal volumes of SAO shock blood, a significant reduction in mean arterial blood pressure was observed. At 90 minutes following transfusion, the mean arterial blood pressure of normal blood transfused animals was ˜85 mmHg, compared to ˜63 mmHg observed in SAO shock blood transfused animals at the same time point. Thus, transfusion of SAO shock blood induced a systemic response in normal acceptor animals that resulted in depression of mean arterial blood pressure, indicating that blood from SAO donors evidently contained cellular activating inflammatory mediators. The descending slope of mean arterial blood pressure values as time progressed may suggest that the inflammatory mediators progressively increased activation of cellular factors in the recipient, in what may have been a pro-inflammatory process. It is possible that the longer these inflammatory mediators circulated throughout the acceptor animals, the more intensely they affected the biological system, as observed by the declining mean arterial blood pressure trace. Based on this result, increasing the observation period post-transfusion might illustrate an even more dramatic mean arterial blood pressure drop.

### Example 9

### Effects of Transfusing Filtered Normal and SAO Shock Blood into Recipients

To design an intervention for treatment of physiological shock, studies are conducted to incorporate testing the ability of the GFF-device to directly filter SAO shock plasma and remove/reduce inflammatory mediators embedded within. Following similar protocols as described above, SAO shock blood is removed from donors, run on a GFF-device, and transfused into a recipient. Monitoring mean arterial blood pressure and survival of the recipient facilitates a method of evaluating the GFF-device. Challenges include blood processing (separating the plasma from the SAO shock whole blood, filtering the plasma, and reconstituting this filtered plasma with the remaining whole blood components) and compensating for plasma volume lost upon filtration. Incorporating protease inhibitor treatment with chemicals such as Futhane may also prove to be beneficial in reversing the effects of physiological shock and increasing survival.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising:
identifying a subject as having or being at risk for an inflammatory disease that is amenable to extracorporeal filtration of a bodily fluid;
inserting a catheter into a selected body part of the subject;
withdrawing the bodily fluid through the catheter;
filtering the bodily fluid to remove one or more mediators of inflammation; and
returning the filtered bodily fluid to the subject.

2. The method of claim 1, wherein the filtering step comprises passing the bodily fluid through one or more glass fiber filters.

3. The method of claim 1, wherein the filtering step comprises passing the bodily fluid through four or more glass fiber filters.

4. The method of claim 1, wherein the filtering step comprises passing the bodily fluid through six or more glass fiber filters.

5. The method of claim 1, wherein the filtered bodily fluid is returned to the subject through the catheter inserted into the selected body part.

6. The method of claim 1, wherein the bodily fluid is blood.

7. The method of claim 6, further comprising separating the blood into cellular and non-cellular components.

8. The method of claim 7, wherein the non-cellular component is filtered and the cellular component is not filtered.

9. The method of claim 8, further comprising recombining the filtered non-cellular component with the unfiltered cellular component prior to returning the blood to the subject.

10. The method of claim 1, wherein the inflammatory mediators are selected from the group consisting of polypeptides, polypeptide fragments, lipids, and lipid fragments.

11. The method of claim 1, wherein the bodily fluid is peritoneal fluid.

12. The method of claim 1, wherein the bodily fluid is lymph.

13. The method of claim 1, wherein the bodily fluid is cerebrospinal fluid.

14. The method of claim 1, wherein the subject is diagnosed as being in shock.

15. The method of claim 1, wherein the subject is diagnosed as having hypertension, diabetes, retinopathy, or Alzheimer's disease.

16. An apparatus comprising:
a bodily fluid removal unit;
an extracorporeal filtration unit operable to remove mediators of inflammation from the bodily fluid, wherein the mediators of inflammation comprise polypeptides, polypeptide fragments, lipids, or lipid fragments; and
a return unit.

17. The apparatus of claim 16, wherein the bodily fluid removal and return units are operable to remove and return blood from a subject.

18. The apparatus of claim 16, wherein the bodily fluid removal and return units are operable to remove and return peritoneal fluid from a subject.

19. The apparatus of claim 16, wherein the bodily fluid the bodily fluid removal and return units are operable to remove and return lymph from a subject.

20. The apparatus of claim 16, wherein the bodily fluid the bodily fluid removal and return units are operable to remove and return cerebrospinal fluid from a subject.

21. The apparatus of claim 16, wherein the filtration unit comprises one or more glass fiber filters.

22. The apparatus of claim 16, further comprising a separator unit operable to separate blood into cellular and non-cellular components.
